(19) United States
(12) Patent Application Publication
(10) Pub. No.: US 2008/0003585 A1
(43) Pub. Date: Jan. 3, 2008

(54) Purification and amplification of nucleic acids in a microfluidic device

(75) Inventor: Luis Ugozzoli, San Rafael, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: BIO-RAD LABORATORIES, INC., a corporation of the state of Delaware, Hercules, CA (US)

(21) Appl. No.: 11/478,953

(22) Filed: Jun. 29, 2006

(51) Int. Cl. C12Q 1/68; C07H 21/04

(52) U.S. Cl. 435/6; 536/25.4

(57) ABSTRACT

Purification of either DNA or RNA and amplification through PCR or reverse-transcriptase PCR are performed in a common microfluidics device by the inclusion of diatomaceous earth in a reservoir within the device and by the use of microfluidics technology for conveying fluids through the reservoir and through microchannels within the device.

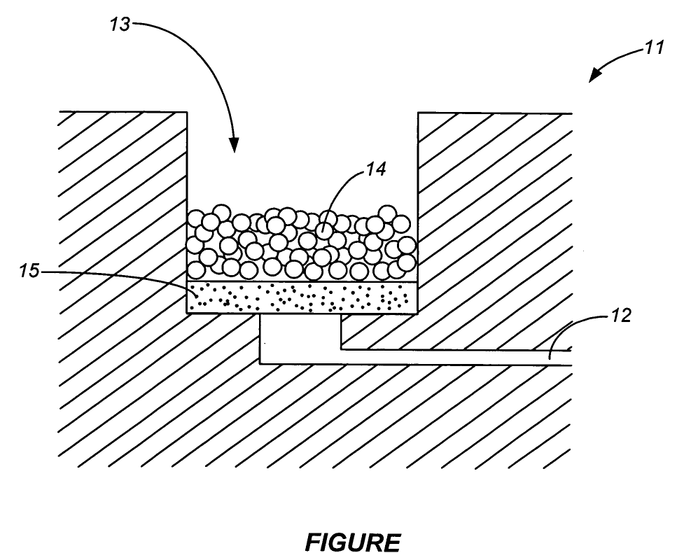

### BACKGROUND OF THE INVENTION

1. Field of the Invention

This invention resides in the field of nucleic acid amplification by the polymerase chain reaction.

2. Description of the Prior Art

The polymerase chain reaction (PCR) is a process for amplifying DNA, i.e., producing multiple copies of a DNA sequence from a single copy or a small number of copies, to facilitate sequence determinations of the DNA. In the typical PCR process, the DNA is combined in a PCR reaction mixture with primers, a DNA polymerase, and other reaction components, and the mixture is heated and cooled through a sequence of temperatures at which the various stages of the process for copying the DNA take place, and the sequence repeated until the desired number of copies is formed. These stages include a high-temperature “melting” of the double-stranded DNA to separate the strands, followed by a relatively low-temperature “annealing” to hybridize the primers to the separated strands, and finally a moderate-temperature primer extension resulting in a double-stranded DNA, identical to the starting DNA, formed from each separated strand. The cycle is then repeated to achieve multiples of the product DNA in sufficient volume to permit analysis. The process is also used in obtaining sequence determinations for RNA, by first synthesizing single-strand DNA that is complementary to the RNA by the use of reverse transcriptase, then amplifying the single-strand DNA for analysis as an analogue of the RNA. Regardless of whether the starting nucleic acid is DNA or RNA, the process will proceed most efficiently and accurately if the starting nucleic acid is in purified form, i.e., DNA fully isolated from RNA, or vice versa, and from all other macromolecular species in the cell lysate in which the nucleic acid originally resides.

Microfluidic devices and microfluidics technology are a well-developed field that involves the use of devices containing networks of microchannels through which chemical or biological samples are conveyed for purposes of performing separations, assays, syntheses, and various other procedures. Disclosures of some of these devices and the technology associated with their construction and use are found in Parce, J. W., et al., U.S. Pat. No. 5,942,443, issued Aug. 24, 1999; Knapp, M. R., et al., U.S. Patent Application Publication No. US 2005/0042639 A1, published Mar. 23, 2006; and other patents and patent application publications assigned to Caliper Life Sciences, Inc. of Mountain View, Calif. The use of microfluidics technology for PCR is disclosed in some of these documents, and also in Kopp, M. U., et al., “Chemical Amplification: Continuous-Flow PCR in a Chip,” Science 280, 1046-1048 (May 15, 1998), and Parthasarathy, R. V., et al., U.S. Patent Application Publication No. US 2005/0142663 A1, published Jun. 30, 2005.

Of further potential relevance to this invention is published material disclosing the use of diatomaceous earth to bind nucleic acids. Examples of this published material are Boom, W. R., et al., U.S. Pat. No. 5,234,809, issued Aug. 10, 1993; Colpan, M., U.S. Pat. No. 6,274,371 B1, issued Aug. 14, 2001; Little, M. C., U.S. Pat. No. 5,075,430, issued Dec. 24, 1991; Vical Incorporated, European Patent Application No. EP 1 291 421 A1, published Mar. 12, 2003; Boom, R., et al., “Rapid and Simple Method for Purification of Nucleic Acids,” J Clin. Microbiol. 28(3): 495-503 (March 1990); Beld, M., et al., “Fractionation of nucleic acids into single-stranded and double-stranded forms,” Nuclic Acids. Res. 24(13): 2618-2619 (1996); Parrish, K. D., et al., “A Rapid Method for Extraction and Purification of DNA from Dental Plaque,” Appl. Environ. Microbiol 61(1), 4120-4123 (1995); Carter, M. J., et al., “An inexpensive and simple method for DNA purifications on silica particles,” Nucleic Acids Res. 21(4), 1044 (1993); and Hansen, N. J. V., et al., “High-Throughput Polymerase Chain Reaction Cleanup on Microtiter Format,” Analytical Biochemistry 296, 149-151 (2001). All of the documents in this and the preceding paragraphs, as well as any others cited in this specification, are incorporated herein by reference.

### SUMMARY OF THE INVENTION

The present invention resides in a microfluidics device that performs both DNA or RNA purification and PCR, and in a method for both purifying the DNA or RNA and amplifying the DNA or a DNA from which the RNA sequence can be determined. In certain aspects of the invention, RNA is separated from DNA in the microfluidics device. In others, DNA is separated from RNA in the microfluidics device. In still further aspects, separation of DNA from RNA or vice versa is performed outside the microfluidics device while further purification of one or the other is achieved in the microfluidics device by separating the nucleic acid from other components of the cell lysate, such as inhibitors and miscellaneous proteins and cell debris. Thus, when the species of interest is DNA, the DNA is purified by isolating it from RNA and other components of the cell lysate in which the DNA resides, the purification being performed either partially or entirely within the microfluidics device. Likewise, when the species of interest is RNA, the RNA is purified by isolating it from DNA and other components of the cell lysate, the purification being performed either partially or entirely within the microfluidics device. All aspects of this invention involve the binding of nucleic acids to diatomaceous earth followed by the elution of the bound nucleic acids from the diatomaceous earth, with at least the elution, and in certain embodiments of the invention both the binding and the elution, occurring within the microfluidics device.

In one group of procedures of this invention, purification begins by contacting the cell lysate with diatomaceous earth under conditions causing all nucleic acids, both DNA and RNA and both single-stranded and double-stranded, in the lysate to bind to the diatomaceous earth. This is done either inside the microfluidics device or prior to placement of the lysate in the microfluidics device. The result is a two-phase solid-liquid medium in which the solid is the diatomaceous earth and bound nucleic acids and the liquid is the remainder of the cell lysate. The two-phase medium is either formed in one of the reservoirs of the device or transferred to one of the reservoirs. In either case, the reservoir is then purged with a wash buffer to remove unbound material. The remaining diatomaceous earth and bound nucleic acids are then incubated with either RNase or DNase, again within the reservoir, followed by another wash buffer to remove the cleaved nucleic acid, and the remaining nucleic acid is then eluted from the diatomaceous earth with an elution buffer.

In other procedures, a particular type of nucleic acid, either DNA or RNA, is selectively bound to the diatomaceous earth, by the use of a binding buffer that is selective for the nucleic acid of choice. This can be done either within or outside of the microfluidics device, but will be followed by elution of the selectively bound nucleic acid in the microfluidics device. In still further procedures, DNA is separated from RNA, or vice versa, by procedures that are performed outside the microfluidics device and do not involve diatomaceous earth, followed first by binding of the separated nucleic acid to, and then elution from, diatomaceous earth for final purification. In these latter procedures, the elution is performed within the microfluidics device.

In each of the embodiments and variations described herein, the final eluted nucleic acid is combined with PCR reagents, or first with reverse transcriptase followed by PCR reagents, and subjected to thermal cycling to effect the PCR process. The integration of nucleic acid purification with PCR in a microfluidics device in accordance with this invention significantly reduces the otherwise time-consuming purification process and allows cell lysates from any tissue in which nucleic acids reside to be placed directly in a microfluidics device. Integration also extends the benefits associated with microfluidics technology to the use of diatomaceous earth as a purification medium for nucleic acids.

Additional objects, advantages, and embodiments of the invention will be apparent from the description that follows.

### BRIEF DESCRIPTION OF THE DRAWING

The FIGURE depicts a microfluidics device of the present invention.

### DETAILED DESCRIPTION OF THE INVENTION AND PREFERRED EMBODIMENTS

Diatomaceous earth (DE), also known as kieselguhr or diatomite, is a loosely coherent chalk-like sedimentary rock consisting primarily of fragments and shells of hydrous silica secreted by diatoms, which are microscopic one-celled algae. The primary component of DE is silica and it is highly porous in structure. DE is commercially available in natural form as well as in calcined and flux-calcined forms. Calcined DE is DE that has been calcined at temperatures in the vicinity of 900-1,000° C., while flux-calcined DE is DE that has been calcined in the presence of soda ash or sodium chloride to reduce the surface area. The various forms of DE are sold under such tradenames as CELITE® (registered trademark of JohnsManville Corp., Lompoc, Calif., USA) and CELATOM® (registered trademark of EaglePicher Filtration and Minerals, Inc., Reno, Nev., USA).

The diatomaceous earth used in the practice of this invention can be in particulate form, or immobilized in a membrane, or packed in porous retainer such as a flow-through cartridge. When in particulate form, the particle size can vary and is not critical to the invention. In preferred embodiments of the invention, the particles are from about 1 μm to about 125 μm in diameter, most preferably from about 5 μm to about 60 μm in diameter.

In procedures in which the first step involves binding all nucleic acids in the lysate to the diatomaceous earth, certain operating conditions will promote the binding, depending on the particular cell lysate and the form of the diatomaceous earth. In certain embodiments of the invention, these conditions include the presence of a chaotropic agent in the solid-liquid medium. Examples of suitable chaotropic agents are guanidinium thiocyanate, guanidinium isothiocyanate, guanidinium hydrochloride, alkali iodides such as sodium or potassium iodide, and alkali perchlorates such as sodium or potassium perchlorate. Guanidinium thiocyanate is preferred. The concentration of the chaotropic agent when present may vary and the precise amount is not critical. The benefit resulting from the presence of the chaotropic agent will generally be achieved at a wide range of concentrations of the agent, with best results generally obtained using concentrations ranging from about 0.8 M to about 10 M. Conditions that promote the binding of the nucleic acids to the diatomaceous earth may also include incubating the solid-liquid medium in the presence of a buffer, preferably one that maintains a pH of from about 6.4 to about 9.5. When both a chaotropic agent and a buffer are used, the chaotropic agent can be combined with the buffer in an aqueous solution which is then added to the solid-liquid medium. An example of a chaotropic agent-containing buffer solution that can be used to bind all nucleic acids to the diatomaceous earth is one whose composition is 6.0 M sodium perchlorate, 0.05 M Tris-Cl pH 8, and 10.0 mM ethylenediamine tetraacetic acid.

In procedures in which selective binding is achieved by the use of a special buffer solution, selectivity can be achieved by a buffer solution that promotes the binding of double-stranded nucleic acids in preference to single-stranded nucleic acids. This will be useful in purifying DNA from mixtures in which RNA is present only in single-stranded form. Procedures and an appropriate buffer solution for achieving this effect are disclosed in the Beld et al. paper referenced above. The selective binding buffer is a lysis/binding buffer whose composition is guanidinium thiocyanate in 0.2M ethylenediamine tetraacetic acid at pH 8.0, prepared by dissolving 120 g of the guanidinium thiocyanate in 100 mL of the EDTA. In procedures where RNA is the nucleic acid of interest, the RNA can be isolated from the DNA by the use of a combination of aqueous and organic solvents disclosed in Chomczynski, P., U.S. Pat. Nos. 4,843,155 and 5,346,994. According to these patents, RNA can be isolated from DNA by an aqueous solvent solution containing phenol and a guanidinium compound at a pH of 4, followed by extraction of the aqueous solution with an organic solvent such as chloroform. The RNA remains in the aqueous phase and is precipitated by adding a lower alcohol prior to being bound to the diatomaceous earth for final purification in the microfluidics device.

Preferred conditions promoting the binding of the nucleic acids to diatomaceous earth in any of the embodiments of this invention are a moderate temperature, preferably from about 15° C. to about 30° C., most preferably about 22° C., and a contact time of from about 3 minutes to about 60 minutes, most preferably from about 5 minutes to about 20 minutes. When a lysate is contacted directly with the diatomaceous earth, the volume ratio of the lysate to the diatomaceous earth can likewise vary, although effective results can be achieved with a volume ratio of approximately 1:1.

The wash buffer used to remove unbound species from the diatomaceous earth after the binding of the nucleic acids can be any buffer that maintains an appropriate pH and separates unbound species from the diatomaceous earth. The wash buffer can also contain the chaotropic agent and can either be the same buffer used to promote the binding of the nucleic acids to the diatomaceous earth or a distinct buffer. In certain embodiments of the invention, the wash buffer contains a lower alcohol such as methanol, ethanol or isopropanol. Ethanol is particularly preferred. When an alcohol-containing buffer is used, the alcohol can constitute from about 20% to about 95% of the buffer on a volume basis. The alcohol-containing wash buffer can also include a salt such as sodium chloride. An example of an alcohol-containing wash buffer that does not contain one of the chaotropic agents listed above is one of the following composition: 20.0 mM Tris-Cl pH 7.5, 2.0 mM EDTA, 0.4 M NaCl, and 50% ethanol. In certain embodiments of the invention, washing is achieved by purging the diatomaceous earth first with a binding buffer, i.e., one that contains a chaotropic agent and a buffer as described in the preceding paragraph, and then with an alcohol-containing buffer that does not contain a chaotropic agent, as described in this paragraph. The total volume of buffer used in this washing step is preferably two or more times the volume of the solid-liquid medium as a whole, and when successive buffers are used, the volume of each is preferably two or more times the volume of the solid-liquid medium.

For procedures that utilize the enzymes RNase and DNase, these enzymes are available commercially from suppliers of chemicals for biological laboratories. Examples of such suppliers are Promega Corporation, Madison, Wis., USA, and Qiagen Inc., Valencia, Calif., USA. Information regarding the optimal amounts or concentrations of the enzyme and the inclusion of any necessary buffers or additives are also available from the suppliers. These and other incubation conditions such as temperature and contact time will also be readily apparent to those skilled in the use of these enzymes.

After the incubation with the appropriate enzyme is completed, removal of the cleaved nucleic acid—RNA in cases where DNA is being purified, and DNA in cases where RNA is being purified—is achieved by a second wash, which can be performed using the same wash buffer or sequence of buffers as used prior to the cleavage or a different wash buffer or buffer sequence. Any buffer that removes unbound nucleic acid and causes no structural transformation of the bound nucleic acid can be used. The environment and operating conditions will be the same as those of the first wash buffer.

Elution of the purified nucleic acid in all embodiments of the present invention is achieved by purging the diatomaceous earth with an elution buffer. Any buffer that will dissociate the nucleic acid from the diatomaceous earth and is otherwise inert to the nucleic acid can be used. The preferred buffer is a low salt buffer, i.e., one with a maximum salt concentration of about 20 mM. A preferred pH range of the buffer is about 7.5 to about 8.5. One example of a low salt buffer is an aqueous solution containing 10.0 mM Tris-Cl pH 8 and 1.0 mM EDTA (ethylenediaminetetraacetic acid). Another example is DEPC (diethylpyrocarbonate)-treated water.

An example of a protocol for RNA preparation is as follows. Whole cells are first mixed with a lysis solution consisting of 4M guanidine thiocyanate, 20 mM Tris, 20 mM EDTA, pH 7, supplemented with 1% mercaptoethanol, either in a tube or within the DE-containing well in the microfluidics device that is designated for sample preparation. An equal volume of 70% ethanol is then added and the mixture is thoroughly mixed, then incubated in the sample preparation well for 1-60 minutes. The liquid phase is then discarded into a waste well and the DE is washed with a low-stringency buffer consisting of 50 mM Tris, pH 7.5 (prepared from a 5× concentrate by dilution with 95-100% ethanol). Unbound material is then discarded into the waste well. DNase I, reconstituted from a lyophilized powder in Tris, pH 7.5, then mixed one part with 15 parts of DNase Dilution Buffer (40 mM Tris-HCI pH 8, 10 mM MgSO₄, 10 mM CaCl₂), is added to the sample preparation well and digestion is allowed to proceed for fifteen minutes at room temperature. The DNase solution is then discarded from the sample preparation well and the DE is first washed with a high-stringency wash buffer consisting of 2.5M guanidine hydrochloride, 10 mM Tris, 10 mM EDTA, pH 7, then the low-stringency wash buffer. The RNA is then eluted with 4-30 μL of DEPC-treated water or T₁₀E₁ elution solution (10 mM Tris, 1 mM EDTA, pH 8.0).

A similar protocol although with RNase instead of DNase can be used for DNA preparation, including a lysis/binding buffer that separates double- from single-stranded nucleic acids. A buffer that can be used for the RNase treatment is 10 mM Tris-HCl, pH 7.5, 5 mM EDTA, 0.3M NaCl.

In embodiments of the invention that utilize diatomaceous earth in particulate form, the solid-liquid medium that includes the diatomaceous earth and the cell lysate or any buffer or wash solution being used at the various stages of the purification procedure will be a suspension of the solid diatomaceous earth particles in the liquid. The suspension in these embodiments can be retained in a reservoir within the microfluidics device by a particle-retaining filter. The filter can be any structure that allows the liquid phase of the suspension to pass while blocking the passage of the diatomaceous earth particles. Examples of such filters are frits, porous membranes, and mesh screens. The pore or aperture size in the filter will be small enough to retain the diatomaceous earth particles. For diatomaceous earth with a particle size range of 5 μm to 60 μm, a preferred filter pore diameter is approximately 3 μm.

Microfluidics devices to which the present invention can be applied are generally those of the type described in the references cited above. As shown in the FIGURE attached to this specification, the typical microfluidics device 11 is characterized by a body structure that contains cavities in the form of microchannels 12 that have at least one dimension that is 500 microns or less, and in many cases 100 microns or less. The typical sample reservoir 13 in which the diatomaceous earth 14 is retained is generally larger than the microchannels that supply the reservoir or draw wash liquid or eluate from it, and a filter 15 as described above retains the diatomaceous earth 14 in the reservoir 13. In most cases, the sample reservoir has a volume ranging from about 5 μL to about 50 μL. The conveyance of liquids into and out of the reservoirs by way of the microchannels can be achieved by any conventional techniques, examples of which are electrophoretic transport, pneumatic transport, and hydraulic transport. One example of a transport system is that disclosed in Boronkay, G., et al., U.S. patent application Ser. No. 11/288,838, filed Nov. 28, 2005. Where necessary, liquids can be directed into particular microchannels and the direction of flow can be reversed or re-directed by conventional methods as well. One example of a method for selecting a liquid flow path among two or more alternative flow paths is the use of electrophoretic forces with selective use of electrodes. Another is the use of pneumatic or hydraulic means in conjunction with microfluidic valves. Such valves are known in the microfluidics art and include rotary valves and diaphragm valves such as those disclosed in Hartshorne, H. A., et al., U.S. Pat. No. 6,748,975 B2, issued Jun. 15, 2004, and bubble valves such as those disclosed in Gilbert, J. R., et al., U.S. Pat. No. 6,877,528 B2.

As noted above, the durations of the various steps of the purification process will be varied and chosen to achieve the optimal result for each step, whether the step be one that involves binding to the diatomaceous earth, the action of an enzyme, washing, or elution. The optimal duration for each step will be known to those skilled in the art or readily determinable by routine experimentation. The selected duration can be achieved by selecting the volume of the particular liquid medium that is being conveyed through a reservoir, the volume of the reservoir, the volumetric flow rate through the reservoir and the microchannels supplying the reservoir, and other parameters and operating conditions of the system.

Once the DNA or RNA purification is completed, PCR reaction materials are added, either directly or following the addition of the reverse transcriptase in the case of purified RNA. These additions and the incubations needed for PCR are performed downstream of the purification stages but within the same microfluidics device. The amplification reactions themselves are likewise performed within the device. The amplification reactions can be performed in the same manner as in the prior art, using reaction mixtures and reaction conditions that are used or have been disclosed for use in these reactions. Means of forming the mixtures, initiating the reactions, and performing them within the microfluidics device can thus be the same as disclosed, for example, in the Kopp et al. paper (Science 1998), the Knapp et al. U.S. Patent Application Publication No. US 2005/0042639 A1, and the Wada et al. U.S. Patent Application Publication No. US 2005/0170362 A1, all cited above. Temperature changes can be effected by maintaining different regions of the microfluidics device at different temperatures by the use of thermoelectric modules or other localized temperature control means, and passing the reaction mixture through the different stages in succession, while durations of exposure to the different temperatures can be controlled by of the manufacture of microchannels of selected lengths in the temperature-controlled sections and the flow rate, the length in each section establishing the residence time of the appropriate reaction mixture at the temperature of that section. Alternatively, thermally cycling can be achieved by heating and cooling the entire microfluidics device to the temperatures required for each stage of the amplification procedure.

The foregoing description is offered primarily for purposes of illustration. Further variations, modifications, and substitutions that can be utilized in implementing the novel concepts of the invention and will therefore fall within the scope of the claims will be apparent to those skilled in the art of PCR, microfluidics, and diatomaceous earth.

What is claimed is:

1. A method for processing a selected nucleic acid from a cell lysate comprising both DNA and RNA in which said selected nucleic acid is one of said DNA and said RNA and the other of said DNA and said RNA is defined as an unselected nucleic acid, to both purify and amplify said selected nucleic acid in a microfluidics device, said method comprising:
(a) forming a mixture comprising said lysate with diatomaceous earth under conditions resulting in the binding of at least a portion of said selected nucleic acid to said diatomaceous earth and the dissolving of all of said unselected nucleic acid in a supernatant;
(b) removing said supernatant from said diatomaceous earth to leave diatomaceous earth with said selected nucleic acid in purified form bound thereto, and eluting said purified selected nucleic acid from said diatomaceous earth;
(c) when said selected nucleic acid is DNA, thermally cycling said eluted DNA in an amplification reaction mixture comprising DNA polymerase and oligonucleotide primers under conditions causing said primers to anneal to complementary target sequences of said eluted DNA and to be extended; and
(d) when said selected nucleic acid is RNA, synthesizing complementary DNA to said eluted RNA using reverse transcriptase and thermally cycling said complementary DNA in an amplification reaction mixture comprising DNA polymerase and oligonucleotide primers under conditions causing said primers to anneal to target sequences of said complementary DNA and to be extended;
steps (a) through (d) being performed within said microfluidics device.

2. The method of claim 1 wherein said selected nucleic acid is DNA, and step (a) comprises contacting said lysate with said diatomaceous earth in the presence of a binding buffer comprising a guanidinium salt at a pH of from about 6.4 to about 9.5.

3. The method of claim 2 wherein said guanidinium salt is guanidinium thiocyanate.

4. The method of claim 1 wherein said selected nucleic acid is RNA, and step (a) comprises contacting said lysate with said diatomaceous earth in the presence of a binding buffer comprising guanidinium salt and phenol at a pH of from about 6.4 to about 9.5.

5. The method of claim 4 wherein said guanidinium salt is guanidinium thiocyanate.

6. The method of claim 1 wherein step (a) comprises:
(a)(i) binding substantially all nucleic acids in said lysate to said diatomaceous earth;
(a)(ii) when purifying DNA, incubating said diatomaceous earth with RNase, and when purifying RNA, incubating said diatomaceous earth with DNase.

7. The method of claim 6 wherein step (a)(i) comprises contacting said lysate with said diatomaceous earth in the presence of a chaotropic agent.

8. The method of claim 7 wherein said chaotropic agent is a member selected from the group consisting of guanidinium thiocyanate, guanidinium isothiocyanate, guanidinium hydrochloride, an alkali iodide, and an alkali perchlorate.

9. The method of claim 7 wherein said chaotropic agent is guanidinium thiocyanate.

10. The method of claim 1 wherein step (b) comprises eluting said purified selected nucleic acid from said diatomaceous earth with an elution buffer of pH 7.5 to 9.5, said elution buffer having a maximum salt concentration of about 20 mM.

11. A method for processing a cell lysate in a microfluidics device by extracting DNA from said lysate, purifying said DNA, and amplifying said purified DNA, said method comprising:
(a) contacting said lysate with diatomaceous earth under conditions causing nucleic acids in said lysate to bind to said diatomaceous earth;
(b) purging said diatomaceous earth with a first wash buffer to remove from said diatomaceous earth components of said lysate that are not bound to said diatomaceous earth;
(c) incubating said diatomaceous earth with RNase and purging said diatomaceous earth with a second wash buffer to remove cleaved RNA;
(d) eluting DNA from said diatomaceous earth by purging said diatomaceous earth with an elution buffer that cleaves bound DNA from said diatomaceous earth; and
(e) thermally cycling said purified DNA so eluted in an amplification reaction mixture comprising DNA polymerase and a primer under conditions causing said primer to anneal to a target sequence of said single strand DNA and to be extended;
steps (b) through (e) being performed within said microfluidics device.

12. A method for processing a cell lysate in a microfluidics device by extracting purified RNA from said lysate and amplifying said purified RNA, said method comprising:
(a) contacting said lysate with diatomaceous earth under conditions causing nucleic acids in said lysate to bind to said diatomaceous earth;
(b) purging said diatomaceous earth with a first wash buffer to remove from said diatomaceous earth components of said lysate that are not bound to said diatomaceous earth;
(c) incubating said diatomaceous earth with DNase and purging said diatomaceous earth with a second wash buffer to remove cleaved DNA;
(d) eluting RNA from said diatomaceous earth by purging said diatomaceous earth with an elution buffer that cleaves bound RNA from said diatomaceous earth; and
(e) synthesizing complementary DNA to said eluted RNA using reverse transcriptase, and thermally cycling said complementary DNA in an amplification reaction mixture comprising DNA polymerase and a primer under conditions causing said primer to anneal to a target sequence of said complementary DNA and to be extended;
steps (b) through (e) being performed within said microfluidics device.

13. A microfluidics device comprising a plurality of reservoirs, diatomaceous earth retained within at least one of said reservoirs, a network of microchannels communicating with said reservoirs, and liquid transport means for conveying liquids through said microchannels and to and from said reservoirs.

14. The microfluidics device of claim 13 wherein said diatomaceous earth is in particulate form, and said microfluidics device further comprises filtration means for preventing passage of said diatomaceous earth, while allowing passage of liquid, from said at least one reservoir.
